(19) United States
(12) Patent Application Publication
(10) Pub. No.: US 2001/0005782 A1
(43) Pub. Date: Jun. 28, 2001

(54) External urinary catheter assembly

(76) Inventors: Allan Tanghoj, Kokkedal (DK); Henrik Lindenskov Nielsen, Smorum (DK); Signe Uhre Guldfeldt, Roskilde (DK)

Correspondence Address: JACOBSON PRICE HOLMAN & STERN, THE JENIFER BUILDING, 400 SEVENTH STREET N W, WASHINGTON, DC, 200042201

(21) Appl. No.: 09/790,652

(22) Filed: Feb. 23, 2001

Related U.S. Application Data
(63) Continuation-in-part of application No. 09/534,199, filed on Mar. 24, 2000.

(30) Foreign Application Priority Data
Nov. 12, 1999 (DK) PA 199901637
Nov. 13, 2000 (DK) PCT/DK00/00629

(51) Int. Cl. A61M 1/00

(52) U.S. Cl. 604/327; 604/331; 604/317; 604/349

(57) ABSTRACT

An external urinary catheter assembly for the relief of male urinary incontinence comprises an inner member (1), which is arrangeable, in use, under the foreskin (6) of a penis and has in its distal end a discharge conduit (5) for connection with a hose, and an outer holder member (8) for circumferential engagement, in use, with the external side of the foreskin (6).

The outer holder member (8) is formed in a distal end with an opening (9) for the discharge conduit (5) of the inner member (1). Connection means (10) is provided to form a stretchable link between the outer holder member (8) and discharge conduit (5) and/or the inner member to maintain the circumferential engagement of the outer holder member (8) with the external side of the foreskin (6) under tensile load conditions acting on the discharge conduit (5), e.g. from the weight of a urine collection bag connected with the discharge conduit (5).

The invention relates to an external urinary catheter assembly for the relief of male urinary incontinence comprising an inner member, which is arrangeable, in a position of use, under the foreskin of a penis in surface contact with the corona thereof, a discharge conduit connected with an opening formed in a distal end section of the inner member to be positioned substantially opposite the urethral orifice in said position of use to provide a sealed urine flow passage extending in a substantially axial direction towards the exterior of the catheter assembly, and an outer holder member formed in a distal end section with an opening for said discharge conduit and arrangeable, in said position of use, in a substantially fixed axial position with respect to the discharge conduit for circumferential engagement with the external side of the foreskin.

External male urinary catheter assemblies of this kind have been suggested in the prior art in a number of different designs.

Thus, from GB-A-2 075 847 an assembly is known comprising a relatively short funnel-like uridom device to be placed directly against the corona of the penis to be kept in place under the foreskin. Around a discharge conduit of the device an external sheathing body is fastened in an inverted position to be brought into engagement with the external side of the foreskin after application of the uridom device to maintain the assembly in place by means of the sandwich position of the foreskin between the uridom device and the external sheathing body.

In use the discharge conduit of the uridom device is connected with a hose member, through which discharged urine flows into a collection bag. After repeated discharges the weight of the collection bag becomes heavier and a tensile load is applied through the hose member and the discharge conduct to the uridom device positioned between the foreskin and the corona. Due to the integral connection of the external sheathing body with the uridom device the tensile load is at least partly transferred also to the sheathing body with the inherent risk that the whole assembly may fall off from its position of use.

GB-A-2 126 483 discloses another design, in which a more safe retainment of the assembly in the position of use is implemented by use of a tubular urine ducting device, e.g. of silicone rubber, arranged outside the corona in extension thereof, so that a rather severe extension of the foreskin is required to keep the tubular device in place by arrangement of an adhesive strip or strap member on the external side of the foreskin. Evidently, the rather severe extension of the foreskin required to keep the tubular device in place causes considerable discomfort in use.

In EP-B-0 706 355 and 0 706 356 external urinary catheter assemblies are disclosed in which a more safe retainment in the position of use is obtained with considerably less discomfort to the user by means of a short inner catheter member designed with improved form stability in its distal end part around the discharge conduit to be more safely kept in place under the foreskin by a separate substantially bowl-shaped outer holder member displaceably surrounding the discharge conduit of the catheter member to be movable between an application position, in which the holder member is partly removed from the catheter member and the position of use.

The use of a separate outer holder member is seen by some users, however, as an inconvenience, as the displaceable arrangement of the holder member on the discharge conduit of the inner catheter member may easily cause complete separation of the holder member from the catheter member during application of the assembly. Whereas this problem may be avoided or at least reduced by a friction fit between the outer holder member and the discharge conduit, this would make the axial displacement of the holder member with respect to the discharge conduit required to bring the holder member from the application position towards the position of use more difficult and cause inconvenience, in particular to tetraplegics or other users having reduced dexterity, For that reason a one-piece assembly would be more attractive to many users.

Moreover, a safe friction fit between the outer holder member and the discharge conduit is needed to maintain the holder member in a fixed axial relationship with respect to the discharge conduit in the position of use and in practical applications of this prior art assembly this requirement has resulted in a tendency for the entire assembly to be detracted from the position of use under tensile load conditions acting on the discharge conduit, e.g. from the weight of a filled or partly filled urine collection bag connected through a hose with the discharge conduit,

It is therefore the object of the present invention to provide a urinary catheter assembly of the kind defined in which the drawbacks of the design of prior art integrated solutions have been eliminated.

According to the invention an external male urinary catheter assembly of the kind defined is characterized in that connection means is provided to form in said position of use a stretchable link either between the outer holder member and the discharge conduit or between the discharge conduit and the inner member to maintain said circumferential engagement of the outer holder member with the external side of the foreskin also under tensile load conditions acting on the discharge conduit.

By provision of said connection means forming a stretchable link between the outer holder member and the discharge conduit the catheter assembly is kept safely in place even under tensile load transferred from the collecting bag through the connecting hose member to the discharge conduit, since a certain movement or stretching of the discharge conduit may be allowed without affecting the position of the outer holder member in safe engagement with the external side of the foreskin. In fact, a tensile load acting on and causing slight stretching or even displacement of the inner member connected with the discharge conduct may increase the holding effect of the outer holder member.

Advantageous and preferred embodiments of the urinary catheter assembly according to the invention are stated in the dependent claims.

In the following the invention will be further explained with reference to the schematical drawings in which

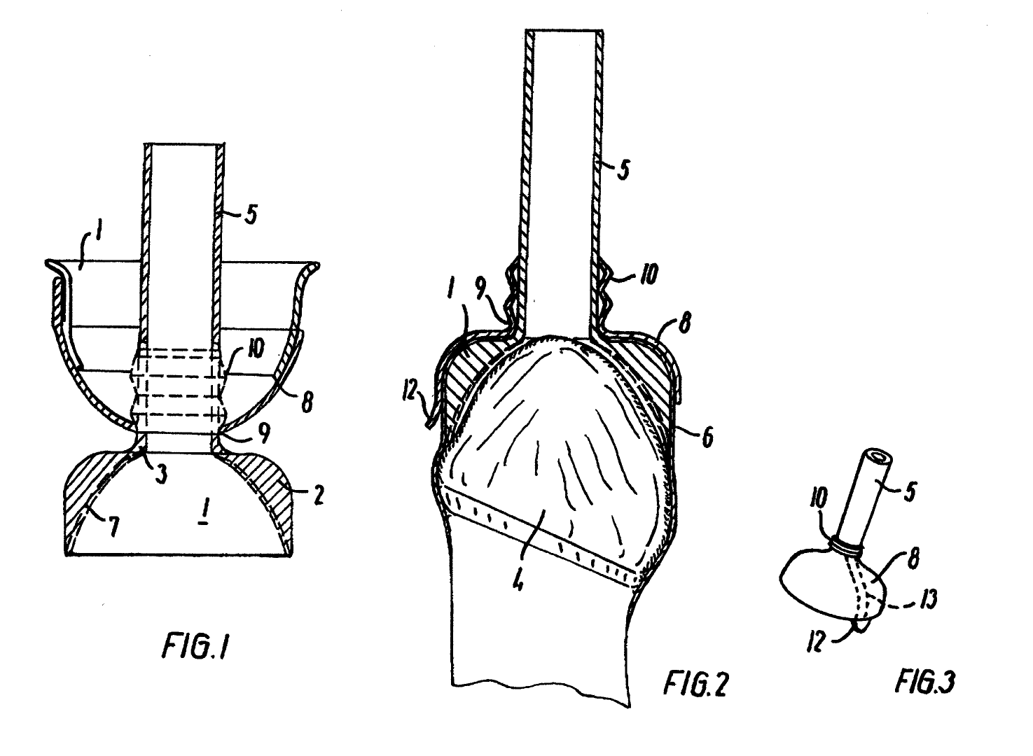

FIGS. 1 and 2 are sectional views of a first embodiment of the catheter assembly in an application position and a position of use, respectively,

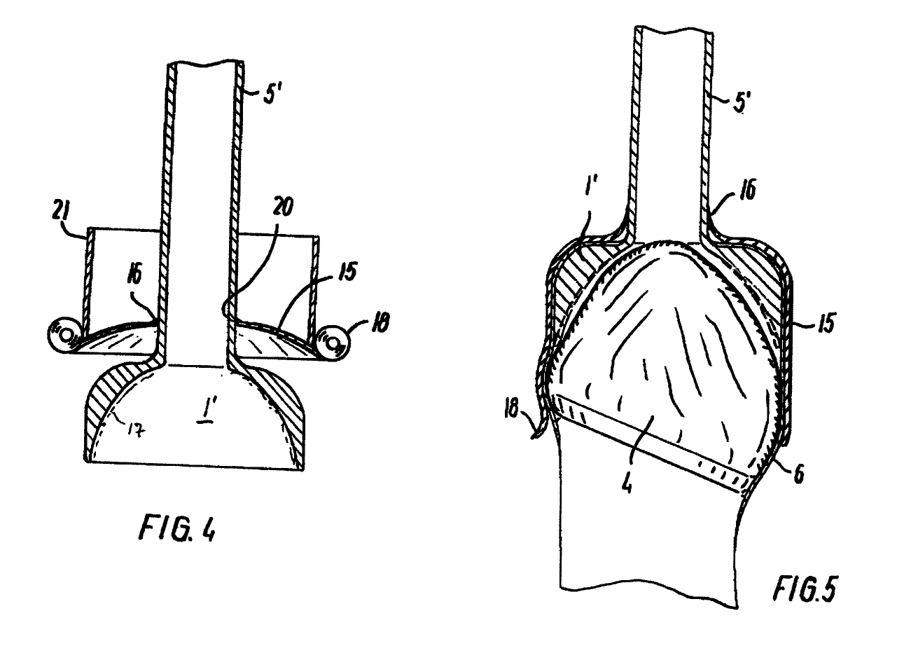

FIG. 3 is a perspective view of the outer holder member in the embodiment in FIGS. 1 and 2, and

FIGS. 4 and 5 are sectional views of a second embodiment in an application position and a position of use, respectively,

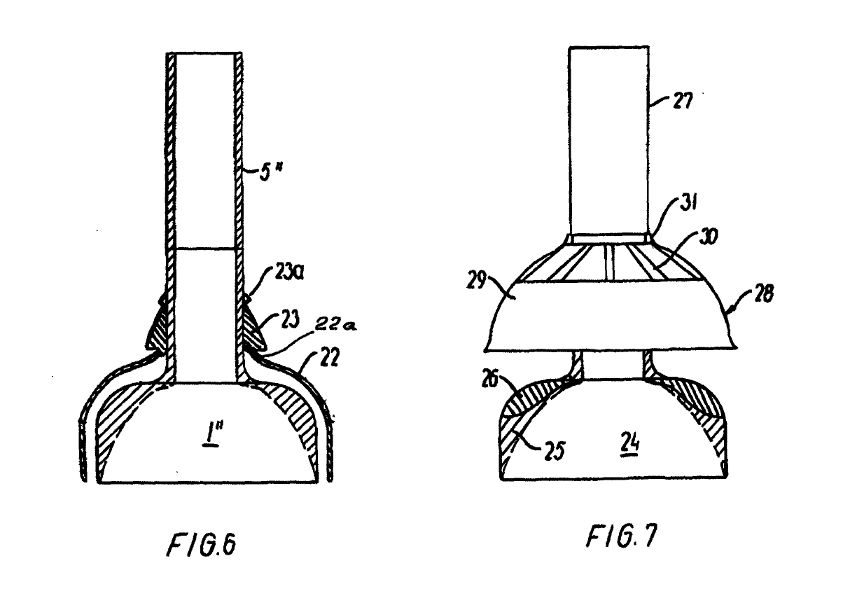

FIG. 6 is a sectional view of a third embodiment,

FIG. 7 a partly sectional view of a fourth embodiment,

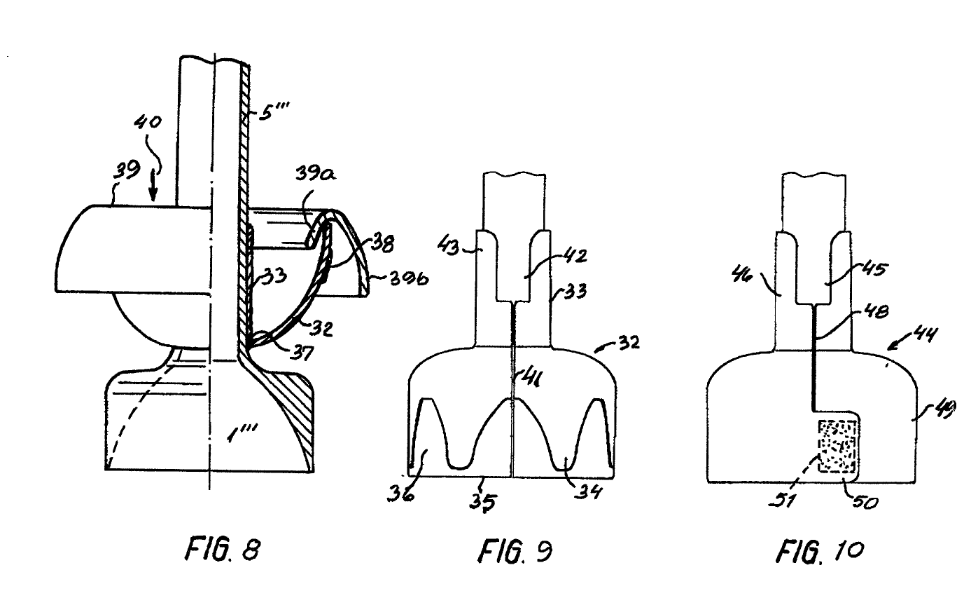

FIG. 8 is a partly sectional view of a fifth embodiment,

FIG. 9 illustrates the embodiment in FIG. 8 in the position of use, and

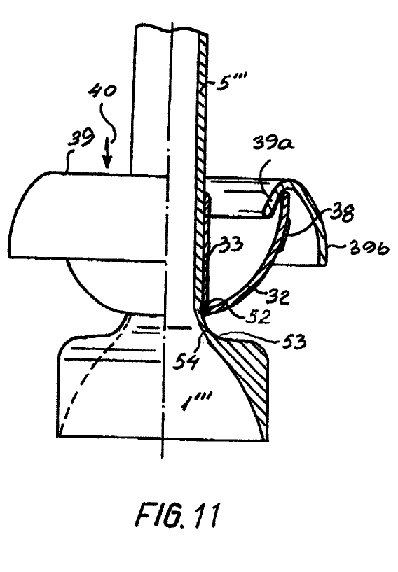

FIG. 10 is a side view of a sixth embodiment.

The catheter assembly shown in FIGS. 1 and 2 comprises an inner member 1 which in a distal end section 2 is formed with an opening 3 which in a position of use as shown in FIG. 2 is opposite the urethral orifice in the corona 4 of a penis. At the opening 3 the distal end section 2 joins a discharge conduit 5 by which the catheter may be connected e.g. with a hose member, through which discharges of urine from the catheter member 1 may flow into a collection bag not illustrated. Thereby a sealed urine flow passage is provided to extend from the inner side of the inner member 1 facing the urethral orifice in a substantially axial direction towards the exterior of the catheter assembly.

As shown in FIG. 2 the inner member 1 is formed to be placed in use under the foreskin 6 of the penis in contact with the corona 4 and the axial length of the part of the inner member contacting the corona is relatively short, e.g. between 5 and 35 mm, such that in use the catheter member only covers the extreme portion of the corona outside its largest diameter, just below which the corona is connected with the foreskin 6 through a sensitive skin band.

In the distal end section 2 around the discharge conduit 5 the catheter 1 may be designed with increased form stability so as to form a shoulder portion having e.g. a substantially flat end face so that in use the catheter member 1 maintains its outer shape in the part which is engaged and retained by the foreskin 6.

As illustrated this increased form stability may be obtained in a simple manner by reinforcement provided by an increased wall thickness in the distal end 2.

The illustrated design of the inner catheter member 1 is known per se from the above mentioned EP patent publications and alternatively the increased form stability may be obtained in any of the ways disclosed in EP-B-0 706 355, the disclosure of which is incorporated herein by reference

The inner member 1 may be made from any material which is suitable for engagement with the sensitive skin of the corona 4 in sealing contact therewith. Suitable materials may comprise thermoplastic elastomeric materials like SBS, SEBS or SIS compounds, polyether, polyester, polyamide or polyurethane and may also comprise thermoplastic materials such as plastizised PVC or curable elastomeric materials like latex, nitrile rubber, chloroprene rubber, SBS rubber, isoprene rubber, silicone rubber or polyurethane.

To provide an improved sealing effect against the corona 4 the inner side of the catheter member 1 may be coated with a layer 7 of a skin-friendly adhesive as used for medical articles intended to contact sensitive skin tissue.

In the embodiment shown the discharge conduit 5 and the inner catheter member 1 are permanently connected with a surrounding outer holder member 8 having an opening 9 for the discharge conduit 5 by connecting means which according to the invention forms a stretchable link. As shown in FIGS. 1 and 2 the connecting means may comprise a short bellows-shaped member 10 having one end connected with the discharge conduit 5 and the other end connected with the outer holder member 8 at the edge of the opening 9.

In the embodiment in FIGS. 1 and 2 the outer holder member 8 is formed as an invertible substantially bowl-shaped member, which from the inverted application position in FIG. 1 may be brought into the position of use by tilting around its transition to the bellows member 10.

The holder member 8 may be made of any suitable soft elastic, semi-rigid or rigid material such as thermoplastic materials or curable elastomeric materials as mentioned above for the inner member 1 or it may alternatively be made in the form of a web of textile material like gauze or non-textile material. For improved engagement with the external side of the foreskin the inner engagement surface of the outer holder member 8 may be coated with a layer 11 of a skin-friendly adhesive as mentioned above for the inner member 1.

Such an adhesive layer may be dimensioned with controlled adhesive properties and/or be applied in various patterns as known in the art to control the adhesive force.

By use of an adhesive layer the length of the outer holder member in the position of use may be reduced to cover only a part of the foreskin in order to increase comfort in connection with removal of the catheter assembly.

The provision of an adhesive is not compulsory, however, and may be dispensed with, since a sufficient holding force may be obtained by other means, e.g. dimensioning of the outer holder member with a length so as to cover, in the position of use the entire external side of the foreskin surrounding the corona.

As will be appreciated from FIG. 2 the stretchable link formed by the bellows-shaped member 10 will function to resist outwards displacement of the outer holder member 8 from its position of use in engagement with the external side of the foreskin 6, when tensile load conditions act as the discharge conduit 2, e.g. from the weight of a collecting bag through a connecting hose member.

Compared to the prior art designs disclosed in the above-mentioned EP patent publications the holding effect of the outer holder member is significantly improved to provide incrased safety also under load conditions resulting from physical activities of the user.

In order to facilitate removal of the catheter assembly after use the proximal end of the outer holder member 8 is formed with a projecting flap 12, which may serve as a grip which can be operated to remove the outer holder member 8 by pulling back the projecting flap 12 in a direction towards the inverted position, from which the holder member was originally applicated.

This feature of the invention may be further improved, if the outer holder member is formed as shown in FIG. 3 with a weakened tear-apart region or zone 13 extending at least partly from the flap 12 towards the opening 9. By operation of the flap 12 the outer holder member 8 will be torn apart for easy removal of the catheter assembly.

As further illustrated in FIG. 1 the inverted application position of the outer holder member 8 may be used for arrangement of an applicator member 14 of a rigid or semi-rigid material inside the holder member 8.

Such an annular applicator will be effective to facilitate accurate positioning of the inner member 1 in contact with the corona during application of the catheter assembly.

In the alternative embodiment shown in FIGS. 4 and 5 the inner member 1′ is of the same design and proportions as shown for the inner member 1 in FIGS. 1 and 2, whereas the outer holder member 15 is formed as a soft flexible uridom-like member which in the application position shown in FIG. 4 assumes a rolled-up condition surrounding the discharge conduit 5′ of the catheter member 1′.

The connecting means providing a stretchable link between the outer holder member 15 and the discharge conduit 5′ is in this embodiment designed as a distal end region 16 of the holder member 15 having reduced wall thickness, but could also comprise a bellows member as shown in FIGS. 1 and 2.

Typically, the holder member 15 will be made of an elastomeric material such as e thermoplastic or curable elastomer as mentioned above. Like in the embodiment of FIGS. 1 and 2 the inner engagement surface of the holder member 15 may optionally be coated with a layer 17 of a skin-friendly adhesive.

Also in this embodiment removal of the holder member 15 after use may be facilitated by forming the proximal end of the holder member 15 with a projecting flap 18 serving as a grip and, like in the embodiment in FIGS. 1 and 2, the holder member 15 may be formed with a weakened tear-apart region or zone 19 extending from the flap 18 at least partly towards the opening 20 for the discharge conduit 5′.

Moreover, the catheter assembly may comprise a separate rigid or semi-rigid annular applicator member 21 of a design known per se arranged in contact with holder member 15 in its rolled-up application position.

In both of the illustrated embodiments the outer holder member 8 or 15 may preferably be formed integrally in one piece with the inner member 1 or 1′ and the discharge conduit 5 or 5′, whereby manufacture of the catheter assembly as a one-piece device may be made simply by moulding. The holder member 8 or 15 may. however, also be connected with the inner member and the discharge conduit by other conventional methods such as welding, adhesive bonding etc.

In FIG. 6 a modification of the embodiment in FIGS. 4 and 5 is shown, in which the inner member 1″ is of the same design as in the embodiments of FIGS. 1-2 and 4-5, whereas the outer holder member 22 is formed as a separate member, not permanently connected with the inner member and the discharge conduit. In its distal end around an opening for the discharge conduit 5″ the holder member 22 joins an annular retaining member 23, which circumferentially engages the discharge conduit 5″ to maintain the holder member 22 in an axially fixed position with respect to the discharge conduit 5″ in the position of use of the catheter assembly.

The holder member 22 shown in FIG. 6 may otherwise be formed as a soft flexible sheathing body of an elastic material as shown in FIGS. 4 and 5 and be connected with the retaining member 23 by a distal end section 22a of reduced wall thickness to provide a stretchable link. Alternatively, a separate holder member may be formed e.g. as a semi-rigid bowl-shaped member connected with a retaining member by a bellows member as shown in FIGS. 1 and 2

The retaining member 23, which may be made from an elastomeric material, may be displaceably arranged on the discharge conduit 5″ and engage it by a relatively close frictional fit to secure the holder member in the axially fixed position in the position of use or the discharge conduit 5″ may be formed with a projecting circumferential rib 23a which can be passed by the retaining member 23 and secures the axially fixed position of the retaining member and thereby the holder member 22.

In the embodiment illustrated in FIG. 7, the inner member 24 is formed with a relatively flat reinforced distal end section 26 in connection with the discharge conduit 27. On the side intended to face the corona of the penis in the position of use a lining 25 of a resilient skin-friendly material joins the formstable distal end section 26. The inner member 24 and the discharge conduit 27 may conveniently be formed as an integral two-component member composed of a soft elastic material such as any of the materials mentioned in the foregoing and a reinforcement material added to or forming the distal end section 26 to provide formstability.

The outer holder member 28 may be formed as shown with a proximal skirt-like part 29 of a semi-rigid elastic material, which is connected with a ring member 31 secured to the discharge conduit 27 by a number of spaced strip-like connecting members 30 providing a stretchable link.

In the embodiment illustrated in FIGS. 8 and 9 and the modification thereof illustrated in FIG. 11 the inner member 1″′ is of the same general form as shown in FIGS. 1-2 and 4-6. The outer holder member is composed of an invertible, bowl-shaped part 32 of a soft elastic, semi-rigid or rigid material, of the same general form as the outer holder member shown in FIGS. 1 and 2, and a generally tubular retaining part 33 formed in one piece with the bowl-shaped part 32 and surrounding the discharge conduit 5″′ over a substantial part of the length thereof. Like the retaining member in FIG. 6 the tubular retaining part 33 may be displaceably arranged on the discharge conduit 5″′ and engage it by a relatively close frictional fit. Alternatively, the tubular retaining part 33 may be connected with the discharge conduit 5″′ by a relatively weak adhesive joint, e.g. localized in one or more dots.

As best seen in FIG. 9 the tubular retaining part 33 and the major part of the adjoining bowl-shaped part 32 is made with an increased wall thickness providing a relief-like surface providing a number of petals 34 towards the free edge 35 of the bowl-shaped part 32, whereas sectors 36 of the bowl shaped part 32 between the petals 34 has a smaller wall thickness. In the embodiment in FIGS. 8 and 9 a local wall thickness reduction at the transition between the retaining part 33 and the bowl-shaped part 32 provides a stretchable elastic link 37 having somewhat the same character and function as a film-hinge, whereby the bowl-shaped part 32 may be brought from the inverted position shown in FIG. 8 to the position of use illustrated in FIG. 9 by snap action.

The flower-like design of the bowl-shaped part 32 with petals 34 has appeared to provide by itself a very good holding effect against the foreskin, which may be further improved by application of adhesive coatings 38 to a part of the side of each petal 34, which in the position of use will engage the foreskin.

In the modification in FIG. 11, in which the tubular retaining part 33 is connected with the discharge conduit 5″′ by an adhesive dot 52, a stretchable link is alternatively formed between the interconnected retaining part and discharge conduit at the joint 52 and the distal end section 53 of the inner member 1″′ by means of a wall part 54 of reduced thickness causing the wall part 54 to collapse by the inversion of the bowl-shaped member 32 towards the position of use, in which the bowl-shaped member 32 is very safely held against the external side of the foreskin due to the combination of the squeezing effect of the flower-like design and the adhesive coatings 38.

As shown in FIGS. 8 and 11 inversion of the bowl-shaped part 32 to the position of use may be assisted by use of an annular applicator member 39 of generally U-shaped cross-section with a relatively short inner wall part 39a and a longer outer wall part 39b. Thereby, the inversion may be performed by a relatively modest pressure on the applicator 39 in the direction of arrow 40.

As further seen from FIGS. 8 and 11 this applicator design also provides a protection of the adhesive coatings 38, which in the inverted position in FIG. 8 are facing outwards.

Removal of the outer holder member illustrated in fis. 8, 9 and 11 may be facilitated by means of linear weakening zones 41 provided at diametrically opposite locations throughout the axial length of the tubular retaining part 33 and the bowl-shaped part 32, each such weakening zone joining the bottom of a recess 42 formed from the free end of the tubular retaining member 33 to provide a pair of gripping flanges 43. By gripping of these flanges 43 the entire outer holder member will be broken at least along one of the weakening zones 41 and may easily be removed from engagement with the foreskin.

In the embodiment illustrated in FIG. 10 the outer holder member 44 is of the same general geometrical shape as shown in FIGS. 8 and 9, but is made of a less rigid, elastic material with uniform relative small wall thickness. From the bottom of one of a pair of recesses 45 formed in the tubular retaining part 46 to provide gripping flanges 47 a section line 48 extending through the axial length of the retaining part 46 and part of the axial length of the bowl-shaped part 49 allows slight separation of the adjoining edge regions. At the free end of the bowl-shaped part 49 a flap member 50 is provided on the inner side with an adhesive coating 51 to secure the bowl-shaped part 49 in engagement with the foreskin in the position of use by an adhesive joint.

1. An external urinary catheter assembly for the relief of male urinary incontinence comprising an inner member (1,1′,1″,1″′,24), which is arrangeable, in a position of use, under the foreskin (6) of a penis in surface contact with the corona thereof, a discharge conduit (5,5′,5″,5″′,27) connected with an opening (3) formed in a distal end section (2) of the inner member (1) to be positioned substantially opposite the urethral orifice in said position of use to provide a sealed urine flow passage extending in a substantially axial direction towards the exterior of the catheter assembly, and an outer holder member (8, 15,22,28,32-33,44) formed in a distal end section with an opening (9, 20) for said discharge conduit (5,5′) and arrangeable, in said position of use, in a substantially fixed axial position with respect to the discharge conduit for circumferential engagement with the external side of the foreskin (6), characterized in that connection means is provided to form in said position of use a stretchable link (10,16,22a,30,37) either between the outer holder member (8,15,22,28,32-33,44) and the discharge conduit (5,5′,5″,5″′) or between the discharge conduit (5,5′,5″,5″′) and the inner member to maintain said circumferential engagement of the outer holder member (8,15,22,287,32-33,44) with the external side of the foreskin also under tensile load conditions acting on the discharge conduit (5,5′,5″,5″′,27).

2. A urinary catheter assembly as claimed in claim 1, characterized in that said distal end section (2) of the inner member (1,1′,1″,1″′) is formed with reinforcing means to provide increased form stability.

3. A urinary catheter assembly as claimed in claim 2, characterized in that said end section (2) of the inner member (1,1′,1″,1″′) forms a shoulder portion providing a substantially flat end face.

4. A urinary catheter member as claimed in claim 2, characterized in that the inner member (24) and the discharge conduit (27) is formed as an integral two-component member composed of a soft elastic material and a reinforcement material added to or forming the distal end section (26).

5. A urinary catheter assembly as claimed in claim 2, 3 or 4, characterized in that said inner member (24) is formed with a lining (25) of a resilient skin-friendly material facing in said position of use the corona of the penis.

6. A urinary catheter assembly as claimed in any of the preceding claims, characterized in that a layer (7,17,38,51) of a skin-friendly adhesive is applied to an inner and/or outer engagement surface of the inner member (1,1′) and/or an inner surface of the outer holder member (32-33,44).

7. A urinary catheter assembly as claimed in any of the preceding claims, characterized in that said connection means comprises a substantially bellows-shaped member (10) having one end connected with the outer holder member (8) at said distal end section and the other end arranged in said position of use in a fixed position with respect to the discharge conduit (5).

8. A urinary catheter assembly as claimed in any of claims 1 to 6, characterized in that the outer holder member (15,22) is formed as a sheathing body of an elastic material and that said connection means comprises an increased stretchability of said distal end section (16,22a).

9. A urinary catheter assembly as claimed in any of the preceding claims, characterized in that the outer holder member (8,15,28), the inner member (1,1′,24) and the discharge conduit (5,5′) are connected with each other.

10. A urinary catheter assembly as claimed in any of the preceding claims, characterized in that the outer holder member (22,32-33,44) is separate from the inner member (1″,1″′) and the discharge conduit (5″,5″′) and is maintained in the position of use in said axially fixed position by an retaining device (23,33,46) circumferentially engaging the discharge conduit (5″, 5″′).

11. A urinary catheter assembly as claimed in claim 10, characterized in that said retaining device (23,33,46) engages the discharge conduit (5″,5″′) by a relatively close frictional fit.

12. A urinary catheter assembly as claimed in claim 10, characterized in that the discharge conduit (5″) is formed with a projecting circumferential rib (23a) for securing said retaining device (23) and thereby the outer holder member (22) in an axially fixed position on the discharge conduit.

13. A urinary catheter assembly as claimed in any of the preceding claims, characterized in that the outer holder member (15,22) is formed as a rolled-up member which can be rolled-out towards its position in use in engagement with the external side of the foreskin.

14. A urinary catheter assembly as claimed in any of claims 1 to 10, characterized in that the outer holder member (8,22,28,32-33) comprises an elastic semi-rigid bowl-shaped device, which is invertible between its position in use and an inverted application position for application and removal of the inner member (1,1″,1″′,24) into and from said position of use.

15. A urinary catheter assembly as claimed in claims 9 and 14, characterized in that said bowl-shaped device forms the outer holder member (8,28).

16. A urinary catheter assembly as claimed in claim 15, characterized in that the outer holder member (28) is formed with a proximal skirt-like part connected with a ring member (31) to the discharge conduit (27).

17. A urinary catheter assembly as claimed in any of claims 10 to 12 and claim 14, characterized in that the outer holder member is composed of a bowl-shaped part (32) and a generally tubular retaining part (33) formed in one piece therewith and surrounding the discharge conduit (5″′) over a substantial part of the length thereof.

18. A urinary catheter assembly as claimed in claim 17, characterized in that said stretchable link (37) is provided by a local wall thickness reduction at the transistion between said retaining part (33) and said bowl-shaped part (32)

19. A urinary catheter assembly as claimed in claim 17 or 18, characterized in that a major part of said bowl-shaped part (32) is made with an increased wall thickness providing a relief-like surface providing a number of petals (34) separated by sectors (36) of a smaller wall thickness.

20. A urinary catheter assembly as claimed in claim 17, 18 or 19, characterized in that linear weakening zones (41) are provided at diametrically opposite locations throughout the axial length of said retaining part (33) and said bowl-shaped part (32).

21. A urinary catheter assembly as claimed in claim 20, characterized in that each of said weakening zones (41) joins a recess (42) formed in said retaining part (33) to provide a pair of gripping flanges (43).

22. A urinary catheter assembly as claimed in claim 17 or 18, characterized in that a section line (48) is formed to extend through the axial length of said retaining part (46) and part of the length of said bowl-shaped part (49) to allow slight separation of adjoining edge regions, and that a flap member (50) having an adhesive coating (51) on its inner side is provided at the bowl-shaped part (49) to secure the outer holder member (44) to the foreskin by a plaster-like joint.

23. A urinary catheter assembly as claimed in any of the preceding claims, characterized in that in a supply condition it comprises an applicator member (14,21,39) arranged in connection with the outer holder member (8,15,32-33) to assist in positioning of the inner member (1,1′,1″′) in its position of use.

24. A urinary catheter assembly as claimed in any of claims 13 or 14 and claim 23, characterized in that the applicator member (14,21,39) is a separate annular member retained by the outer holder member (8,15,32-33) in its rolled-up or inverted position.

25. A urinary catheter assembly as claimed in claim 24, characterized in that the applicator member (39) has a substantially U-shaped cross-section to provide an outer wall (39b) providing protection of an adhesive coating (38) on said bowl-shaped device (32).

26. A urinary catheter assembly as claimed in any of claims 1 to 14, characterized in that the outer holder member (8,15,32-33) is formed with a projecting flap or flange member (12,18,43) serving as a grip for removal of the outer holder member (8,15,32-33) from its position of use.

27. A urinary catheter assembly as claimed in claim 26, characterized in that said projecting flap member (12,18) joins a weakened tear-apart zone (13,19) extending through at least part of the outer holder member (8,15).
